(19) United States
(12) Patent Application Publication
(10) Pub. No.: US 2020/0132539 A1
(43) Pub. Date: Apr. 30, 2020

(54) Device for Measuring a Distribution of Pressure Across a Surface and Calibrating the Measurement using a Total Mass Measurement

(71) Applicants: David R. Hall, Provo, UT (US); K. Jeffrey Campbell, Spanish Fork, UT (US); Joshua Larsen, Spanish Fork, UT (US); Jared Reynolds, Pleasant Grove, UT (US); Vivek Garg, Murray, UT (US)

(72) Inventors: David R. Hall, Provo, UT (US); K. Jeffrey Campbell, Spanish Fork, UT (US); Joshua Larsen, Spanish Fork, UT (US); Jared Reynolds, Pleasant Grove, UT (US); Vivek Garg, Murray, UT (US)

(21) Appl. No.: 16/173,171

(22) Filed: Oct. 29, 2018

(51) Int. Cl. G01G 19/50; A61B 5/053; A61B 5/00; E03D 11/00

(52) U.S. Cl. CPC G01G 19/50; A61B 5/0537; A61B 2562/046; E03D 11/00; A61B 2562/0261; A61B 5/7275

(57) ABSTRACT

The device includes a pressure-sensing pad which measures a load distribution across its surface and a rigid layer which measures total mass. The mass measurement may be used as a ground-truth measurement with which to calibrate the measurements collected by the pressure-sensing pad. Accordingly, a more accurate pressure distribution may be created using calibrated pressure distribution data. The pressure-sensing pad may include an array of sensors which may be in connection with a flexible layer. When pressure is applied to any of the sensors within the array of sensors, the flexible layer may compress and change the capacitance at the sensors on which the pressure is applied. The rigid layer may include strain gauges which measure the total mass on the device. A controller may include instructions for performing calculations to convert the measurements into a pressure map using calibrated measurements of the pressure distribution across the pressure-sensing pad.

### BACKGROUND

### Field of the Invention

This disclosure relates to diagnostic devices, devices for measuring mass and pressure, and uses thereof.

### Background of the Invention

Pressure pads with regular arrays of sensors are used to acquire the distribution of a user's mass over the surface area beneath the user's feet. This information may be used to provide information about the user's posture, indicate compensation for an injury, and other measurements that are relevant to the subject's health. However, current technologies lack the accuracy, precision, and stability to accurately calculate the user's total mass and provide an accurate pressure map which indicates the distribution of pressure over the bottom surface of the user's feet while standing. Accordingly, such devices are lacking in their diagnostic ability.

### BRIEF SUMMARY OF THE INVENTION

We disclose a device for measuring pressure distribution across a surface and total mass. The device may include both a pressure-sensing pad and a rigid layer which may be adhered to one another. In an example, the pressure-sensing pad may be laminated to the rigid layer. The pressure-sensing pad may include an array of sensors, each sensor including a piezoresistive deposition which is configured to measure pressure applied to a defined point. The array of sensors may be incorporated into a flexible layer which may compress when a user stands on it. The compression may cause a change in capacitance at the sensors on which the pressure is applied. Consequently, analysis of the changes in capacitance through the sensors within the sensor array may be used to create a pressure map which describes the pressure distribution across the surface of the pressure-sensing pad.

The rigid layer may include one or more strain gauges. In some embodiments, the rigid layer may include a plurality of strain gauges, for example, 4, 6, or 8 strain gauges. The rigid layer may measure a total mass placed on the device, for example the total mass of a user standing on the device. The total mass may be used as a ground-truth measurement against which to calibrate the measurements the pressure-sensing pad collects.

Some embodiments may include a plurality of electrodes which may collect bioimpedance measurements from a user who may be standing on the device. In an example, the bioimpedance measurements may be used to calculate a percent body fat of the user.

In some embodiments, the device may include a controller which may be in electronic connection with the pressure-sensing pad and the rigid layer. The controller may also be in electronic connection with the plurality of electrodes which may collect bioimpedance measurements.

The controller may include non-transitory computer readable medium which may store instructions for creating the pressure map from the measurements collected by the pressure-sensing pad and for calculating the total mass using the measurements taken by the strain gauges in the rigid layer. The non-transitory computer readable medium may also store instructions for calculating a calibrated pressure map in which the total mass is used as a ground-truth measurement against which to calibrate the measurements from the pressure-sensing pad. The non-transitory computer readable medium may also store instructions for performing calculations using the bioimpedance measurements collected by the plurality of electrodes. In an example, the instructions may include an algorithm for calculating a user's percent body fat.

In some embodiments the non-transitory computer readable medium may include instructions for creating a postural assessment of a user standing on the device. In some embodiments, the controller may include a memory which may store measurements taken when a user stands on the device. The non-transitory computer readable medium may include instructions for identifying changes in the postural assessment between the user's sessions with the device. For example, improvement in response to therapy and the impact of an injury may be assessed. The non-transitory computer readable medium may also include instructions for identifying trends in the user's posture. For example, if a user tends to put more weight on one side than the other, the device may detect this repeated tendency. Such measurements may indicate a weak or injured side of the user's body or a skeletal abnormality.

In some embodiments, the device may include a user identification function. For example, the memory on the controller may store a user's first set of measurements. The non-transitory computer readable medium may store instructions for comparing the first set of measurements with subsequent measurements to identify a user. The user identification function may enable the controller to track changes in measurements collected from each user over time as well as trends in repeated measurements taken from each user.

In some embodiments, the device may be in connection with an external medical device. In an example, the external medical device may be a medical toilet which collects additional measurements which may be relevant to the user's health. In some embodiments, the controller may be housed within the device and in other embodiments, the controller may be housed external to the pressure-sensing pad and rigid layer. In an example, the controller may be disposed within the medical toilet to which the device is connected.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings.

FIG. 1A illustrates a perspective view from an aerial vantage of an embodiment of the pressure- and mass-measuring device according to an embodiment of the disclosure.

FIG. 1B illustrates a perspective view of the device of FIG. 1A from beneath the device.

FIG. 2 illustrates an exploded view of the device of FIGS. 1A and 1B.

FIG. 3 illustrates a perspective view of an embodiment of the pressure- and mass-measuring device according to an embodiment of the disclosure which includes bioimpedance sensors.

FIG. 4 illustrates a perspective view of the pressure- and mass-measuring device in connection with a medical toilet according to an embodiment of the disclosure.

### DETAILED DESCRIPTION OF THE INVENTION

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “electronic” and “electronically” mean either wired or wireless. For example, the phase “in electronic communication” means either a wired communication between two devices or could mean a wireless communication between devices, such as Wi-Fi.

As used herein, “bioimpedance” means bioelectrical impedance, body impedance, or electrical impedance of the body.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

We disclose a device which collects measurements which may be used to calculate a relative distribution of load across the system and calibrate these measurements using a ground-truth mass measurement, the total mass measurement. The device may include a pressure-sensing pad which may include an array of sensors. Each sensor within the array of sensors may include a piezoresistive deposition which may measure pressure applied to a defined point. The pressure measurement may be collected by measuring a change in capacitance at each sensor in response to pressure. In this way, the piezoresistive depositions may act as pressure-sensing points on the pressure-sensing pad. Together, sensors within the array of sensors may create a pressure map of the total mass distribution across the pressure sensing pad.

The pressure-sensing pad may include a flexible layer within which or on which the array of sensors is disposed. The flexible layer may also be extensible and elastic. Consequently, the pressure-sensing pad may give in response to pressure, thus changing the capacitance at each sensor on which the pressure is applied.

In some embodiments, the pressure-sensing pad further comprises a plurality of primary tracks and a plurality of secondary tracks. Each of the plurality of primary tracks and the plurality of secondary tracks may be constructed from a conductive ink or paste and the piezoresistive depositions may be disposed on the plurality of primary tracks and in connection with at least one of the plurality of secondary tracks. The conductive ink or paste may be an extensible and elastic and may be printed on the flexible layer using any printing technique known in the art. In this embodiment, the multiple primary tracks may transmit a printed electrical signal. In some embodiments, the secondary tracks may also comprise an extensible and elastic conductive ink or paste and be printed using any printing technique known in the art. In some embodiments, the device may further include a controller and each of the pluralities of primary and secondary tracks may be in electronic connection with the controller without crossing each other. In some embodiments, the pressure-sensing pad may be as described in U.S. Pat. No. 9,032,804, titled “Large-area extensible pressure sensor for textiles surfaces,” filed on Jul. 3, 2013, which is hereby incorporate by reference in its entirety.

The device may also include a rigid layer that includes at least one strain gauge. In some embodiments, the rigid layer includes a plurality of strain gauges, for example 4, 6, or 8 strain gauges. By integrating one or more strain gauges into the device along with the pressure-sensing pad, the device simultaneously gathers total mass and load distribution across the surface of the device. Consequently, the one or more strain gauges may provide a measurement which may be used to calibrate the measurements collected by the pressure-sensing pad using a ground-truth mass measurement.

The pressure-sensing pad and the rigid layer may be adhered to each other using a variety of methods known in the art. In some embodiments, the pressure sensing pad may be adhered to the rigid layer by laminating the pressure-sensing pad to the rigid layer.

In some embodiments, the device includes a plurality of electrodes. The plurality of electrodes may be in connection with the pressure-sensing pad and configured to measure bioimpedance of a user who is standing on the device. In some embodiments, the plurality of electrodes may be laminated to the surface of the pressure sensing pad.

In some embodiments, the device includes a controller. The array of sensors and the at least one strain gauge may be in electronic communication with the controller. The controller may include non-transitory computer readable medium. The non-transitory computer readable medium may store instructions for mapping changes in capacitance of the array of sensors in response to pressure and for calculating a total mass placed on the device. In these embodiments, the non-transitory computer readable medium may also include instructions for using the total mass measurement to calibrate a plurality of measurements collected by the pressure-sensing pad.

In some embodiments, the non-transitory computer readable medium stores instructions for creating a postural assessment of a user standing on the device. This may be accomplished by analyzing a pressure map created by the algorithms stored on the controller and then applying algorithms also stored on the controller which assess the user's posture at the time the measurements were taken. The controller may also include a memory which stores one or more sets of measurements the device collects in response to a user standing on the device. The non-transitory computer readable medium may store instructions for identifying changes in the postural assessment between the user's multiple sessions with the device as well as trends in the user's posture as he or she stands on the device. These changes may be used to assess improvement in response to a therapeutic treatment or the impact of an injury over time. In addition, the non-transitory computer readable medium may store instructions for identifying trends in the user's posture which may indicate an injury or skeletal abnormality.

In some embodiments, the plurality of electrodes which may measure bioimpedance may be in electronic communication with the controller. The non-transitory computer-readable medium on the controller may store instructions for calculating a user's percent body fat using a measurement collected by the plurality of electrodes. This calculation may include the measurement of total mass collected by the stain gauges.

In some embodiments, the device may be in connection with an external medical device. The controller may use measurements from the disclosed device in combination with those collected by the external medical device to perform calculations that are relevant to a user's health. In some embodiments the external medical device may be a medical toilet. The medical toilet may collect measurements that are relevant to the user's health as the user sits on or otherwise interacts with the toilet. For example, the toilet may include a device which measures a user's heart rate, measures a user's body temperature, takes electrocardiogram measurements, measures volume and content of a user's urine or feces, measures urine flow rate, identifies changes to a user's skin, and analyzes volatile organic compound in the headspace of the toilet bowl that originate from a user's urine, feces, or flatulence. The medical toilet may include a bidet system which, not only cleanses a user, but includes sensors which collect some of the measurements listed above.

In some embodiments, the controller may be internal to the pressure-sensing pad, the rigid layer, or both the pressure-sensing pad and the rigid layer. In other embodiments, the controller is external to both the pressure-sensing pad and the rigid layer. In the latter embodiment, the controller may be in electronic connection with the pressure-sensing pad and the rigid layer through electrical wires or through wireless mechanisms known in the art. In embodiments in which the device is in connection with an external medical device, the controller may be disposed within the external medical device. For example, in embodiments in which the disclosed device is in connection with a medical toilet, the controller may be disposed within the medical toilet. The controller may receive measurements from both the disclosed device and the medical toilet.

Some embodiments may include a user identification function. In these embodiments, the non-transitory computer-readable medium may store a first set of measurements taken from a user. The non-transitory computer-readable medium may further store instructions for identifying the user each subsequent time the user stands on the device by comparing the current set of measurements to the first set of measurements. By identifying each user, the device may collect and store multiple measurements from each user and identify changes in the measurements for each user over time as well as trends in the measurements over time. For example, the non-transitory computer readable medium may include instructions for identifying the user and for identifying changes in the postural assessment between the user's sessions with the device. For example, improvement in response to therapy and the impact of an injury may be assessed. The non-transitory computer readable medium may also include instructions for identifying the user and for identifying trends in the user's posture or weight distribution. For example, if a user tends to put more weight on one side than the other, the device may detect this repeated tendency. Such measurements may indicate a weak or injured side of the user's body or a skeletal abnormality.

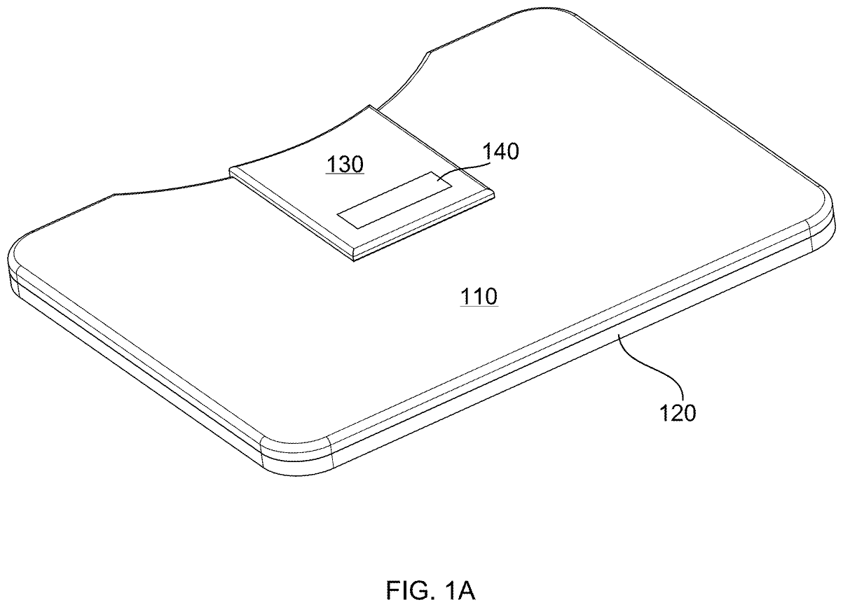

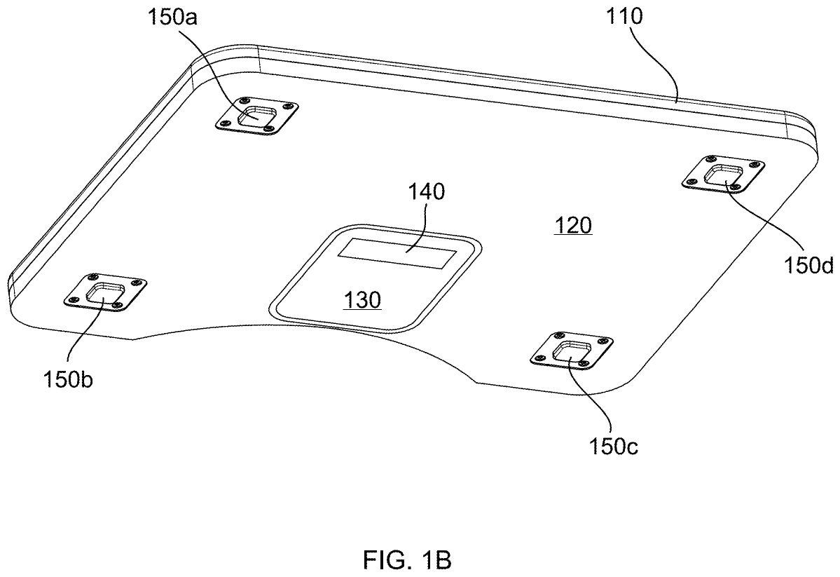

Referring now to the drawings, FIG. 1A illustrates an embodiment of the disclosed pressure- and mass-measuring device. The device includes a top layer which is pressure-sensing pad 110 and a bottom layer which is rigid layer 120. In this embodiment, pressure-sensing pad 110 and rigid layer 120 are laminated to each other. Pressure-sensing pad 110 provides information about relative distribution of load across the system. More specifically, the user steps on the device and the array of sensors in pressure-sensing pad 110 change their capacitance in response to the pressure from the user's feet. Rigid layer 120 includes four strain gauges which, together, measure the total mass on the device. Housing 130 is connected to rigid layer 120 and encloses electronics that are included in the pressure- and mass-measuring device. The electronics include controller 140 which is in electronic connection with the array of sensors within pressure-sensing pad 110 and the strain gauges within rigid layer 120.

FIG. 1B illustrates the embodiment of FIG. 1A from below the device. Housing 130 with controller 140 within are visible from below as they are in connection with rigid layer 120. Strain gauges 150a, 150b, 150c, and 150d are embedded within rigid layer 120 and are in electronic connection with controller 140.

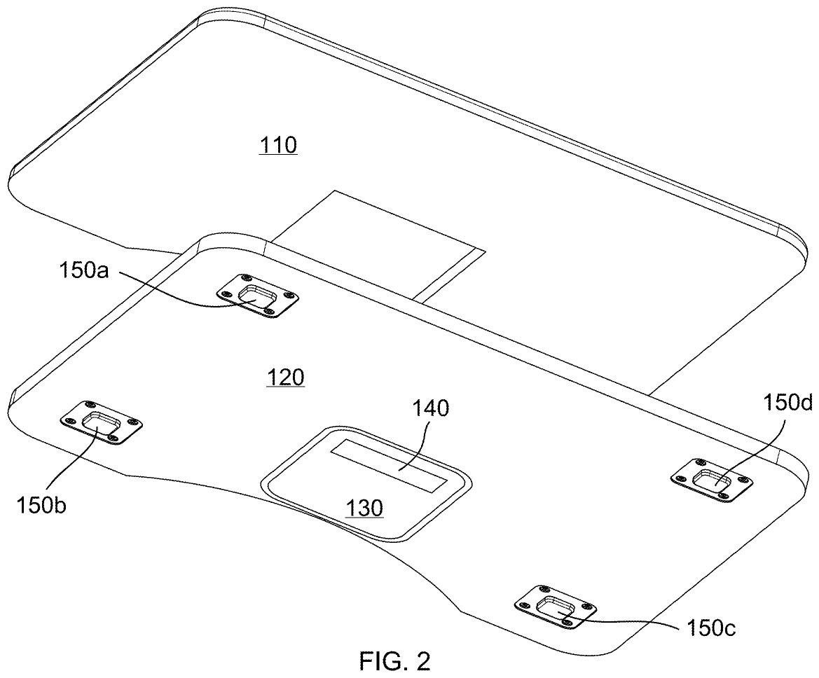

FIG. 2 illustrates pressure-sensing pad 110 and rigid layer 120 separated from each other. In this embodiment, housing 130 is in connection with rigid layer 120. When pressure-sensing pad 110 and rigid layer 120 are adhered together, housing 130 is inserted through a cut-out area of pressure-sensing pad 110.

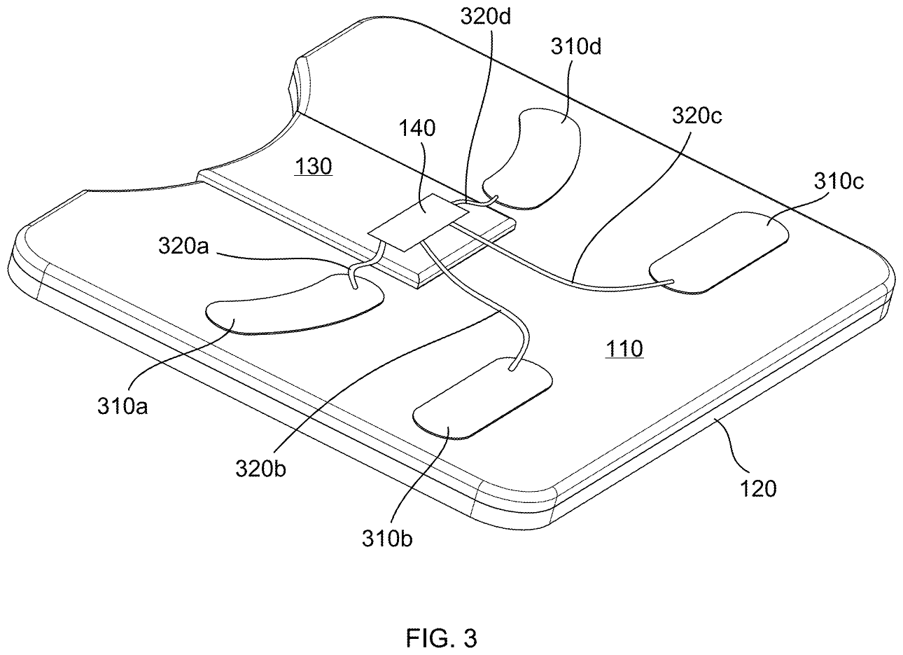

FIG. 3 illustrates another embodiment of the disclosed pressure- and mass-measuring device which also includes pressure-sensing pad 110 and rigid layer 120. Unlike the embodiment of FIGS. 1A, 1B, and 2, the embodiment of FIG. 3 includes electrodes 310a, 310b, 310c, and 310d which may collect bioimpedance measurements as a user stands on them. Electrodes 310a-d are in electronic communication with controller 140 through wires 320a, 320b, 320c, and 320d respectively. Thus, in addition to the pressure map and total mass measurements the device collects, electrodes 310a-d also collect bioimpedance measurements from the user as the user stands on the device and algorithms stored on controller 140 may include these measurements in calculations which provide data relevant to the user's health.

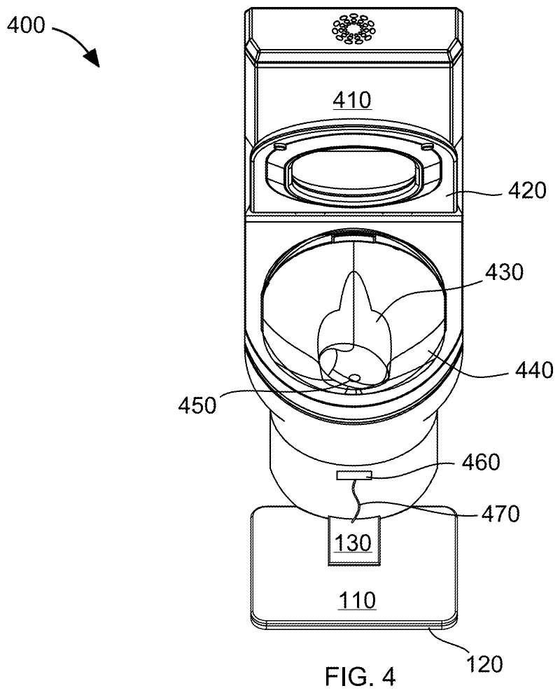

FIG. 4 illustrates the embodiment of the device shown in FIGS. 1A and 1B as part of medical toilet 400. Like traditional toilets, medical toilet 400 includes tank 410, toilet seat 420, and toilet bowl 440. Medical toilet 400 also includes urine capture reservoir 430 which includes orifice 430. A user may urinate into toilet bowl 440 and at least a portion of the urine may collect in urine capture reservoir 430. The urine may then flow through orifice 430 which may lead to analytical equipment which may conduct measurements of urine analytes and other characteristics of the user's urine. This analysis of the user's urine may provide information which may be relevant to the user's health. In some embodiments, the analytical equipment may include one or more of infrared, near-infrared, ultraviolet, visible spectroscopy, and flow cytometry.

Medical toilet 400 includes controller 460 which is in electronic connection with the device through electrical wire 470. In this embodiment, the array of sensors in pressure-sensing pad 110 and the strain gauges in rigid layer 120 are in electronic connection with controller 460 through electrical wire 470. Algorithms stored within controller 460 perform calculations which provide data which may be relevant to the user's health.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

1. A device for measuring pressure distribution and total mass comprising:
a. a pressure-sensing pad comprising an array of sensors, each sensor comprising a piezoresistive deposition which is configured to measure pressure applied to a defined point; and
b. a rigid layer comprising at least one strain gauge;
c. wherein the pressure-sensing pad is adhered to the rigid layer; and
d. wherein the device is configured to measure pressure distribution through the array of sensors in the pressure-sensing pad and total mass through the strain gauges in the rigid layer.

2. The device of claim 1, wherein the pressure-sensing pad comprises a flexible layer.

3. The device of claim 1, wherein the pressure-sensing pad is adhered to the rigid layer by laminating the pressure-sensing pad to the rigid layer.

4. The device of claim 1, further comprising a plurality of electrodes, wherein the plurality of electrodes is in connection with the pressure-sensing pad, and wherein the plurality of electrodes is configured to measure bioimpedance.

5. The device of claim 1, further comprising a controller;
a. wherein the array of sensors is in electronic communication with the controller,
b. wherein the at least one strain gauge is in electronic communication with the controller; and
c. wherein the controller comprises non-transitory computer readable medium which stores instructions for mapping changes in capacitance of the array of sensors in response to pressure and for calculating a total mass placed on the device.

6. The device of claim 5, wherein the controller is external to the pressure-sensing pad and the rigid layer.

7. The device of claim 5, wherein the non-transitory computer readable medium comprises instructions for creating a postural assessment of a user standing on the device.

8. The device of claim 7, wherein the non-transitory computer readable medium stores instructions for storing a user's postural assessment and for identifying changes in the postural assessment between the user's sessions with the device.

9. The device of claim 7, wherein the non-transitory computer readable medium stores instructions for storing a user's postural assessment and for identifying trends in the postural assessment.

10. The device of claim 5, wherein the non-transitory computer readable medium stores instructions for using the total mass measurement to calibrate a plurality of measurements collected by the pressure-sensing pad.

11. The device of claim 5, wherein the non-transitory computer readable medium stores a first set of measurements taken from a user; and further comprises instructions for identifying the user each subsequent time the user stands on the device.

12. The device of claim 5, further comprising a plurality of electrodes, wherein the plurality of electrodes is in connection with the pressure-sensing pad, and wherein the plurality of electrodes is configured to measure bioimpedance.

13. The device of claim 12, wherein the plurality of electrodes is in electronic communication with the controller.

14. The device of claim 13, wherein the non-transitory computer readable medium stores instructions for calculating a user's percent body fat using a measurement collected by the plurality of electrodes.

15. The device of claim 5, wherein the device is in connection with an external medical device.

16. The device of claim 15, wherein external medical device comprises a medical toilet, wherein the medical toilet collects measurements relevant to a user's health.

17. The device of claim 15, wherein the controller is internal to the medical toilet.

18. The device of claim 1, wherein the at least one strain gauge comprises either 4, 6, or 8 strain gauges.

19. The device of claim 1, wherein the pressure-sensing pad further comprises a plurality of primary tracks and a plurality of secondary tracks, wherein each of the plurality of primary tracks and the plurality of secondary tracks comprise a conductive ink or paste, and wherein the piezoresistive depositions are disposed on the plurality of primary tracks and in connection with at least one of the plurality of secondary tracks.

20. The device of claim 19, further comprising a controller, and wherein each of the pluralities of primary and secondary tracks are in electronic connection with the controller without crossing each other.
